(19) United States
(12) Patent Application Publication
(10) Pub. No.: US 2018/0112174 A1
(43) Pub. Date: Apr. 26, 2018

(54) RETROFIT DEVICES FOR BIOREACTORS

(71) Applicant: Medlmmune Limited, Cambridge (GB)

(72) Inventors: William HOLMES, Cambridge (GB); Kenneth LEE, Cambridge (GB); Marcel KUIPER, Cambridge (GB)

(21) Appl. No.: 15/571,313

(22) PCT Filed: May 5, 2016
(86) PCT No.: PCT/EP2016/060130

§ 371 Date: Nov. 2, 2017

Related U.S. Application Data
(60) Provisional application No. 62/157,529, filed on May 6, 2015.

(51) Int. Cl. C12M 1/36; C12M 1/34; C12M 1/26; B01L 3/00

(52) U.S. Cl. CPC C12M 41/48; C12M 41/44; C12M 33/07; B01L 3/567; G05D 11/133; B01L 2200/0605; B01L 2200/02; B01L 2200/12; B01L 2400/0666

(57) ABSTRACT

Disclosed herein are a retrofit device adapted to upgrade bioreactor systems that require at least partially manual addition of liquids, and a method for using the retrofit device with the bioreactor systems. The retrofit device automatically adds the liquids by weight to the bioreactor vessels according to a protocol or procedure. The retrofit device also includes a valve that in the event of power cut-off automatically closes to prevent unintentional addition of liquids.

### FIELD OF THE INVENTION

This invention generally relates to devices that retrofit conventional bioreactor systems (comprising a bioreactor vessel containing probes and sensors, and a bioreactor controller) that require at least some manual controls of the addition of liquids into the bioreactor vessels. The retrofit device provides automatic, precise addition of liquids without or with limited intervention by operators.

### BACKGROUND OF THE INVENTION

Bioreactors are devices or systems that support biological active environments or devices used to culture or grow cells or tissues. Bioreactors can be run in different modes of operation such as batch, fed-batch, or continuous. Bioreactors generally comprise a disposable or autoclavable reaction vessel with inlets for adding substances such as liquids or air, sensors for measuring temperature, pH, or dissolved oxygen, etc., and outlet(s) for sampling and harvesting the cultured cells or tissues. The vessels may be any type of container including tanks or bags. Bioreactor tanks typically have an agitation system with rotational impellers or up-down baffles to keep the liquids inside thoroughly mixed. Bioreactor bags are typically affixed to platforms that are rocked back and forth about one or more axis.

When biological organisms, such as microorganisms or cells, are grow in bioreactor systems, liquids and gasses are added to the bioreactor vessels through ports or inlets. The environmental conditions within the vessels, including temperature, nutrient concentrations, pH, dissolved oxygen for aerobic fermentations, and other dissolved gases, are monitored and controlled. The heat from the bio-reactions, particularly highly exothermic fermentations, can be managed by heat exchangers, such as cooling coils. Liquids can be added to the bioreactor vessels in multiple bolus additions, or in fed-batch systems, or continuous systems, and the pH of the content inside the vessels is measured and can be adjusted by the addition of pH modifiers such as acid/CO₂ or base. For aerobic reactions and for some anaerobic reactions, oxygen or air may be added.

In conventional bioreactors liquids are manually added to the system. Manual additions are currently performed using calibrated peristaltic pumps with volumes added based on pump speed and pump running time; or by using syringes with filters; or by generating positive pressure on liquids in a bottle by using a syringe attached to the bottle. Each manual method has its own disadvantages.

For example, incorrect pump speed or incorrect pump running time with the peristaltic pumps can add incorrect amounts of liquids to the bioreactor vessels. Additionally, clipped tubing, tubing that has lost its elasticity, incorrect pump tubing, pump failure, or loss of calibration can cause incorrect additions by these pumps. Operator errors can cause addition of incorrect materials or amounts when using syringes, and there is a potential for filter failure to occur when excessive force or pressure is applied on the filters, which typically have 0.2 μm pores.

These issues can be remedied by using automated liquid additions of pre-determined amounts of liquid at pre-determined time intervals during a bioreactor run or culture. However, there is no available device that can retrofit and update existing bioreactor systems. Hence, there is a need in the art for such retrofitting and updating devices.

### SUMMARY OF THE INVENTION

Hence, the invention is directed to stand-alone retrofit devices that will perform automated liquid additions to existing bioreactor vessels. Preferably, the retrofit devices are low cost and can extend the useful lives of existing bioreactor systems, which can be expensive to replace.

The invention is also directed to bioreactor systems that have been retrofitted to automatically add liquids based on the decrease in weight or mass of the reservoirs containing the liquids to be added as measured by load cells. The reservoirs containing the liquids to be added may be any type of vessel or container such as a bag or bottle. Measuring the added liquids by the change or decrease in weight of the reservoirs containing the liquids obviates the possible disadvantages associated with adding liquids by volume as described above.

According to another aspect of the present invention, stand-alone retrofit automated liquid addition devices can be used to upgrade any type of bioreactor system, and any machinery that requires the addition of liquids from time to time, such as brewing equipment and the like.

According to another aspect of the present invention, the retrofit device should prevent the unintentional addition of liquids in case of a power loss.

The present invention relates to a method to retrofit a bioreactor system that requires at least partially manual addition of liquids to the bioreactor vessel. The method comprises connecting a retrofit device to said bioreactor system including the steps of

(a) supporting at least one reservoir containing a liquid on a load cell;

(b) connecting the load cell to a microprocessor in the retrofit device, wherein the load cell transmits a signal relating to the weight of the reservoir(s) containing a liquid to the microprocessor;

(c) positioning a tubing from a reservoir containing a liquid to be opened and closed by an electrical valve, wherein depending on said signal the microprocessor selectively moves the electrical valve to open or to close the tubing connected to the selected reservoir containing a liquid;

(d) aseptically coupling the tubing to the bioreactor.

The microprocessor preferably, selectively moves the electrical valve to open or to close depending on a pre-determined decrease of the weight of the at least one reservoir containing a liquid. The electrical valve preferably comprises a solenoid valve, and preferably further comprises a compression spring disposed around an axle of the solenoid valve. The microprocessor preferably opens the solenoid valve by withdrawing the axle and compresses the compression spring. The microprocessor may close the solenoid valve by cutting off electrical power to the solenoid valve and the compression spring returns the axle to close the valve.

The inventive method may further include a step of seeking a user input concerning the pre-determined decrease in the weight of the reservoir containing a liquid before step (a), or a step of displaying information relating to the retrofit device. Optionally, a plurality of additional reservoirs containing liquid can be loaded to the load cell.

The present invention further relates to a retrofit device adapted to be connected to and to be used with a bioreactor system that requires at least partially manual addition of liquids to the bioreactor vessel. The retrofit device may comprise a housing, a microprocessor electrically connected to at least one load cell adapted to measure a weight of at least one reservoir containing a liquid, and an electrical valve controlled by the microprocessor, wherein depending on a signal relating to the weight of the at least one liquid reservoir from the load cell the microprocessor selectively moves the electrical valve to open or to close a tubing, said tubing is aseptically coupling the liquid reservoir to the bioreactor vessel.

Preferably, the electrical valve comprises a solenoid valve. The electrical valve may comprise a compression spring disposed around an axle of the solenoid valve. The microprocessor may open the solenoid valve by withdrawing the axle and compressing the compression spring. The microprocessor can close the solenoid valve by cutting off electrical power to the solenoid valve and the compression spring returns the axle to close the valve. The retrofit device may comprise at least one manual control disposed on the housing to receive a user input relating to a pre-determined decrease in weight of the reservoir containing a liquid.

The present invention may also relate to a combination of a retrofit device and a bioreactor system that requires at least partially manual addition of liquids to the bioreactor vessel, wherein the retrofit device is adapted to be connected to and to be used with said bioreactor system

### BRIEF DESCRIPTION OF THE DRAWINGS

In the accompanying drawings, which form a part of the specification and are to be read in conjunction therewith and in which like reference numerals are used to indicate like parts in the various views:

FIG. 1 is a schematic drawing of the retrofit device illustrated with only one reservoir containing a liquid and tubing for clarity;

FIG. 2 is an enlarged view of the normally closed electrical valve; and

FIG. 3 is a perspective view of an exemplary housing of the retrofit device shown in FIG. 1.

### DETAILED DESCRIPTION OF THE PREFERRED EMBODIMENTS

The present invention relates to a stand-alone retrofit device that can be attached to and to be used with existing bioreactor systems. The retrofit device comprises a microprocessor connected to at least one load cell, which load cell is connected to at least one reservoir containing a liquid, and measures the weight of the liquid in each reservoir. This provides the retrofit device with the ability of adding liquids to the bioreactor vessels based on weight. Preferably, one load cell is paired with at least one reservoir containing a liquid. When the weight of a reservoir is decreased by a desired amount, i.e., a pre-determined weight of the liquid has been dispensed, the retrofit device stops the liquid dispensing from this reservoir. One load cell may be connected to multiple reservoirs, and can measure several liquid additions through the sequential scheduling of their additions.

Measuring the added liquids by the change or decrease in weight of the reservoirs containing a liquid obviates the possible disadvantages associated with adding liquids by measured volume, as described above. Measuring the added liquids by measuring the change or decrease in weight of a reservoir containing a liquid is also preferred over measuring individually each amount of liquid to be added, although measuring each individual addition of liquid is within the scope of the present invention. When the weights of individual additions are small, they may require highly sensitive load cells or scales to accurately measure the small changes in weight.

Suitable load cells include, but are not limited to, strain gauge load cells which comprise strain gauges typically in groups of four arranged in a “Wheatstone bridge” formation and which measure the weight placed on them or hung from them to provide an electrical signal indicative of the weight. The electrical signal can be read and processed by a controller or a microprocessor. Any microprocessor can be used including the Arduino Uno processor, the Raspberry Pi processor or more complex processors. The microprocessor also controls electrical valves that control the liquid additions. The valves are preferably attached to tubings that connect the liquid reservoirs to the bioreactor vessel. Suitable valves include valves that are controllable by the microprocessor, such as solenoid valves and other electrical or electronic valves. Maintenance of an aseptic fluid path is important for cell culture.

In one embodiment, the microprocessor can instruct the valve to advance an axle to a first position directly obstructing the tubing to stop the flow of liquid and to allow the liquid to flow from the reservoir containing a liquid to the bioreactor vessel. In a preferred configuration, a spring is provided that holds an axle to normally pinch the tubing to keep it closed and a solenoid valve is attached to the spring to compress the spring to move the axle away from the tubing to open the tubing and allow flow of liquid. In this preferred configuration, if power is cut-off the solenoid valve automatically releases the spring and the axle will pinch the tubing to prevent unintentional dispensing of liquids when power is disrupted.

The microprocessor is programmable to dispense liquids into the bioreactor vessel according to any protocol or procedure. Preferably, the microprocessor comprises a user interface, preferably a graphical user interface (GUI), or a computer screen to allow the operators to program a new protocol or to select a pre-programmed protocol. The protocol will have information related to the weight amounts of the different liquids and the time intervals to add the liquids to the bioreactor vessel. The liquids are transferred from the reservoir containing a liquid to the bioreactor vessel using gravity, in most cases.

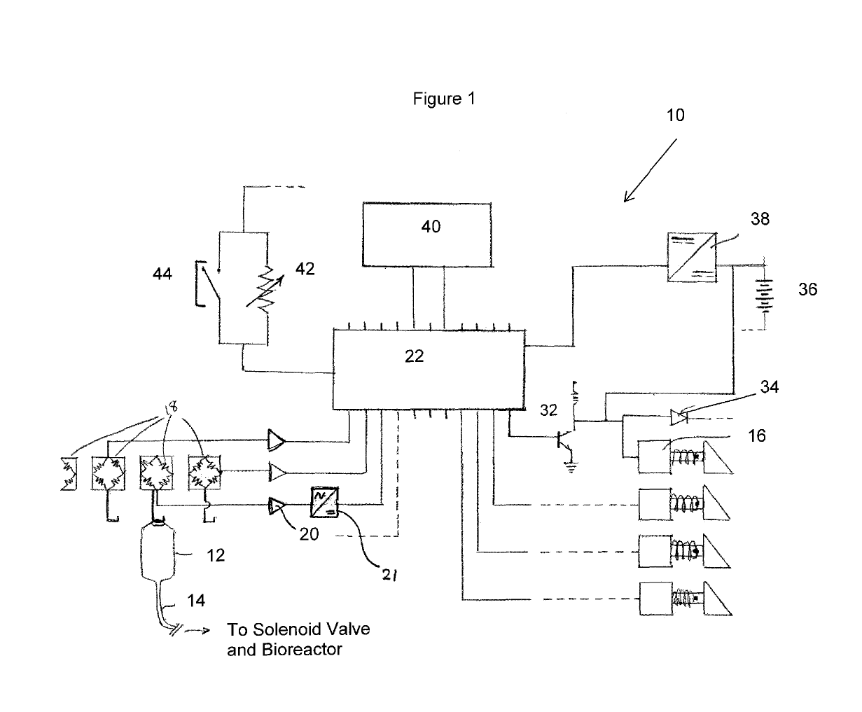

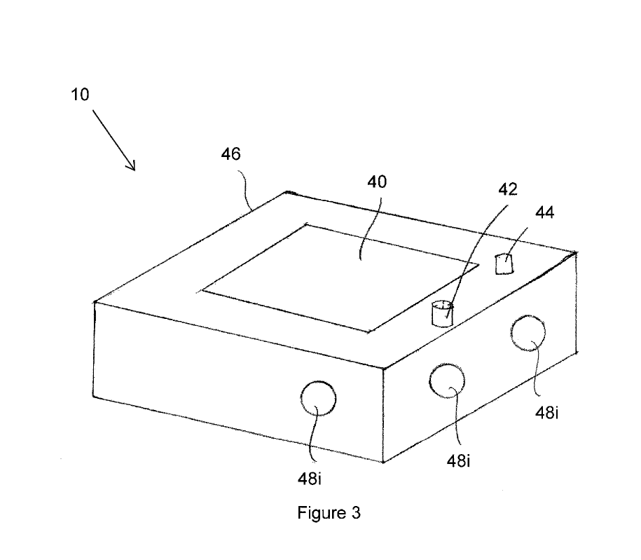

Referring to FIG. 1, retrofit device 10 is shown with only one reservoir containing a liquid 12 and tubing 14 for clarity. Any number of reservoirs containing liquid can be used with retrofit device 10. The liquid from reservoir 12 is transported through a solenoid valve 16, which pinches tubing 14 to stop flow or releases tubing 14 to allow flow described below, to a bioreactor. A housing of retrofit device 10 is omitted for clarity in FIG. 1, but the retrofit device would normally be stored inside a housing or casing as illustrated in FIG. 3. As shown, reservoir 12 is suspended from or supported on load cell 18, which preferably is a strain-gage load cell described above. Load cell 18 continually weighs reservoir 12 as the liquid flows from the reservoir, and load cell 18 produces an electrical signal representing the decrease in weight of reservoir 12. This electrical signal may be amplified by amplifier 20 before being received and processed by microprocessor 22. Optionally, an analog digital converter 21 (ADC) may be incorporated between the amplifier 20 and microcontroller 22 to give a higher resolution signal. A 24 bit ADC may be used, although if a slightly lower resolution is acceptable, an 18 bit ADC may be used, if resolution is not a consideration, an 8 to 10 bit ADC may be used.

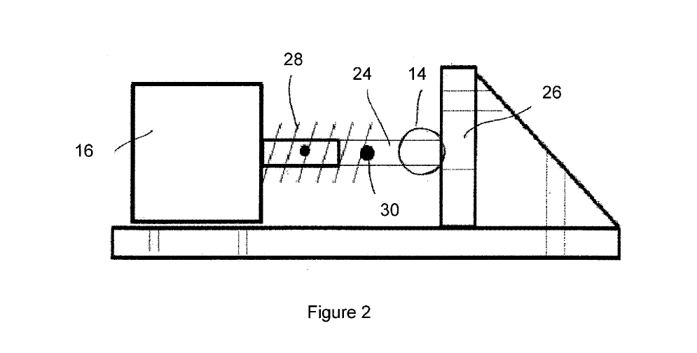

Referring to FIG. 2, tubing 14 from reservoir 12 passes between axle 24 of solenoid valve 16 and stop 26. As shown, tubing 14 is simply superimposed on axle 24 to illustrate the position of tubing 14. When valve 16 is in the closed position as shown in FIG. 2, the wall of tubing 14 would be deformed or pinched by axle 24 to close tubing 14. Return spring 28 surrounds axle 24 and is held between the housing of valve 16 and a knob 30 on axle 24. When microprocessor actuates or runs electricity to valve 16, axle 24 and knob 30 move to the left as the valve is oriented in FIG. 2 thereby compressing spring 28 between knob 30 and the housing of the valve. Tubing 14 is fully open allowing the liquid feed from reservoir 12 to flow therethrough. To close the flow, microprocessor 22 cuts-off power to valve 16 and spring 28 pushes axle 24 and knob 30 to the right pinching tube 14 between axle 24 and stop 26 to cut-off the flow through tubing 14.

Preferably, valve 16 is a fail-safe valve. Spring 28 is sized and dimensioned to push axle 24 toward stop 26 to pinch tubing 14, when power is cut-off to retrofit device 10 to prevent unintentional feeding to the bioreactor vessel. In another embodiment, microprocessor 22 can send a signal to move axle 24 to the left to open tubing 14 and send another signal to move axle 24 to the right to pinch tubing 14.

Referring back to FIG. 1, retrofit device 10 may have a transistor 32 and/or diode 34 connected to each valve 16. Retrofit device 10 can be powered by a battery 36. Since solenoid valves 16 and microprocessor 22 and the other sensors and components may require different voltages and currents, one or more DC/DC converter 38 can be used. As shown, the voltage and current from battery 36 can be used directly by valves 16, and the voltage of battery 36 can be stepped down to a lower voltage by converter 38 to be used by microprocessor 22 and the other sensors and components. Alternatively, retrofit device 10 can be plugged into a wall socket and the AC current from the wall socket is converted to DC current by an AC/DC converter.

Transistors 32 are preferably used as amplifiers. Microprocessor 22 may send a low voltage signal to the transistor, which amplifies the signal to the higher voltage used by solenoid valves 16. Transistors 32 may also be used as electrical switches to turn valves 16 ON or OFF. Diodes 34 are preferably light emitting diodes (LEDs), and each diode 34 may be lighted when a corresponding individual solenoid valve 16 is activated to indicate which liquid feed reservoir 12 is feeding the bioreactor. Diodes can also operate as a one-way current flow restrictors and may be used for that purpose in the retrofit devices of the invention.

Microprocessor 22 may be connected to a screen 40, preferably a touch-screen, that serves as a GUI for an operator to enter a new protocol or select the protocol to operate the bioreactor system. Additional controls can be provided by variable resistor or rotary potentiometer 42 and/or pushbutton switch 44. The operator can specify the weights for each liquid feed addition and time intervals when to start the additions by using the rotary potentiometer 42 and/or pushbutton 44 to select values and move through the menu displayed on screen 40.

FIG. 3 illustrates an exemplary embodiment of retrofit device 10 within housing 46. Preferably, screen/GUI/touchscreen 40 is visible and accessible to the operators, as well as pushbutton selector 44 and rotatable potentiometer 42 to allow the operators to select the weights and start times of the liquid additions and to monitor the operations of the retrofit device. In one embodiment, the components shown in FIG. 1 are enclosed within housing 46. In other embodiments, one or more of the battery 36, load cell(s) 18, solenoid valves 16, and stops 26 can be located outside of housing 46. Ports 48i can be adapted to electronically or electrically connect one or more of these externally positioned components to the rest of retrofit device 10.

As illustrated in FIG. 3, retrofit device 10 is a modular or stand-alone device that can be used with any existing bioreactor system, including but not limited to the autoclavable or disposable bioreactor systems manufactured by Applikon® Biotechnology, the WAVE™ Bioreactor series manufactured by General Electric Healthcare Life Sciences and the HyClone™ Bioreactor from Thermo Scientific, among others. In some embodiments, retrofit device 10 can simply be positioned proximate to the existing bioreactor system(s) and take advantage of the bioreactor's load cells and reservoirs containing liquid. Retrofit device 10's solenoid valves 16 and stops 26 can be positioned around tubings 14 before they are connected to inlet ports on the bioreactor system. Alternatively, tubings 14 or portion(s) or loop(s) thereof can be inserted into slots in housing 46, wherein these slots correspond internally to be between axle 24 of solenoid valves 16 and stops 26 maintaining an aseptic fluid transfer path.

The operation of retrofit device 10 is described with reference to FIG. 1. Multiple liquid feed reservoirs 12 are loaded onto load cells 18. Load cells 18 read the initial weight of each reservoir 12 and send the readings in the form of electrical signals through optional amplifiers 20 to microprocessor 22. Within microprocessor 22 resides software that contains instructions relating how much weights of each liquid feed are to be added to the bioreactor and at what starting times. Microprocessor 22 sends an electrical instruction to the valve 16 that corresponds to the specific reservoir 12 from which liquid is to be dispensed. Said electrical instruction is amplified by transistor 32 and is applied to valve 16. As the selected reservoir is dispensed, the weight of that reservoir 12 decreases which can be displayed in real time on screen 40. After a predetermined weight of the liquid has been dispensed, microprocessor either sends another electrical instruction to valve 16 to close or can stop sending any electrical signal to transistor 32/valve 16. This cessation of electrical signal would cut-off power to valve 16 and compressed spring 28 is released to push axle 24 to pinch tubing 14 to stop the dispensing of liquid. Preferably, the next addition to the bioreactor is paused until the just dispensed liquid is thoroughly mixed inside the bioreactor. Thereafter, the next liquid from another reservoir 12 is dispensed in the same manner, until all liquids in the protocol are added to the bioreactor vessel. No manual input from the operator is necessary.

While only one reservoir 12 and four load cells 18 with four valves 16 are shown, retrofit device 10 may have as many load cells 12 and valves 16 as necessary, and preferably several reservoirs containing liquid are suspended from one load cell. Alternatively, the reservoir containing a liquid is positioned on top of the load cell. Alternatively, one single load cell can measure the changes in weight of a plurality of reservoirs containing liquid 12.

The present inventors' insight is to upgrade existing bioreactor systems that require at least some manual liquid additions with a retrofit device. Bioreactor systems are expensive to replace and presently there is no commercial retrofitting device available.

While it is apparent that the illustrative embodiments of the invention disclosed herein fulfill the objectives stated above, it is appreciated that numerous modifications and other embodiments may be devised by those skilled in the art. Therefore, it will be understood that the appended claims are intended to cover all such modifications and embodiments, which would come within the spirit and scope of the present invention.

We claim:

1. A method to retrofit a bioreactor system that requires at least partially manual addition of liquids to the bioreactor comprising connecting a retrofit device to said system including the steps of
(a) supporting at least one reservoir containing a liquid on a load cell;
(b) connecting the load cell to a microprocessor in the retrofit device, wherein the load cell transmits a signal relating to the weight of the reservoir(s) to the microprocessor;
(c) positioning a tubing from the reservoir to be opened and closed by an electrical valve, wherein depending on said signal the microprocessor selectively moves the electrical valve to open or to close the tubing connected to the selected reservoir;
(d) aseptically coupling the tubing to the bioreactor.

2. The method of claim 1, wherein the microprocessor selectively moves the electrical valve to open or to close depending on a pre-determined decrease of the weight of the at least one reservoir containing liquid.

3. The method of claim 1, wherein the electrical valve comprises a solenoid valve.

4. The method of claim 3, wherein the electrical valve further comprises a compression spring disposed around an axle of the solenoid valve.

5. The method of claim 4, wherein the microprocessor opens the solenoid valve by withdrawing the axle and compresses the compression spring.

6. The method of claim 5, wherein the microprocessor closes the solenoid valve by cutting off electrical power to the solenoid valve and the compression spring returns the axle to close the valve.

7. The method of claim 1 further comprising supporting a plurality of additional reservoirs containing liquid to the load cell.

8. The method of claim 2 further comprising a step of seeking a user input concerning the pre-determined decrease in the weight before step (a).

9. The method of claim 1 further comprising a step of displaying information relating to the retrofit device.

10. A retrofit device adapted to be connected to and to be used with a bioreactor system that requires at least partially manual addition of liquids to the bioreactor vessel comprising:
a housing;
a microprocessor electrically connected to at least one load cell adapted to measure a weight of at least one reservoir containing liquid;
an electrical valve controlled by the microprocessor, wherein depending on a signal relating to the weight of the at least one reservoir containing a liquid from the load cell the microprocessor selectively moves the electrical valve to open or to close a tubing, wherein the tubing is aseptically coupling the liquid reservoir to the bioreactor vessel.

11. The retrofit device of claim 10, wherein the electrical valve comprises a solenoid valve.

12. The retrofit device of claim 11, wherein the electrical valve further comprises a compression spring disposed around an axle of the solenoid valve.

13. The retrofit device of claim 12, wherein the microprocessor opens the solenoid valve by withdrawing the axle and compresses the compression spring.

14. The retrofit device of claim 13, wherein the microprocessor closes the solenoid valve by cutting off electrical power to the solenoid valve and the compression spring returns the axle to close the valve.

15. The retrofit device of claim 10 further comprising a screen that displays information relating to the retrofit device.

16. The retrofit device of claim 10 further comprises at least one manual control disposed on the housing to receive a user input relating to a pre-determined decrease in weight of the reservoir containing a liquid.

17. A combination of a retrofit device and a bioreactor system that requires at least partially manual addition of liquids to the bioreactor vessel, wherein the retrofit device is adapted to be connected to and to be used with said bioreactor system, wherein the retrofit device comprises:
a housing;
a microprocessor electrically connected to at least one load cell adapted to measure a weight of at least one reservoir containing a liquid;
a solenoid valve controlled by the microprocessor, wherein depending on a signal relating to the weight of a liquid reservoir from the load cell the microprocessor selectively moves the solenoid valve to open or to close a tubing, wherein the tubing is aseptically coupling the liquid reservoir to the bioreactor vessel.

18. The combination of claim 17, wherein a compression spring is disposed around an axle of the solenoid valve.

19. The combination of claim 18, wherein the microprocessor opens the solenoid valve by withdrawing the axle and compresses the compression spring, and wherein the microprocessor closes the solenoid valve by cutting off electrical power to the solenoid valve and the compression spring returns the axle to close the valve.
